(19) United States
(12) Patent Application Publication
(10) Pub. No.: US 2006/0141460 A1
(43) Pub. Date: Jun. 29, 2006

(54) Assays for fprl-1 ligands

(75) Inventors: Duncan Henderson, Leicesterhire (GB); Sylvia Salter, Leicestershire (GB); Aram Elagoz, Montreal (CA); Eric Grazzni, Montreal (CA); Paola M. C Lembo, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca A B

(21) Appl. No.: 10/528,009

(22) PCT Filed: Sep. 19, 2003
(86) PCT No.: PCT/GB03/04132

§ 371 Date: Jan. 17, 2006

Related U.S. Application Data
(60) Provisional application No. 60/412,026, filed on Sep. 19, 2002.

(51) Int. Cl. C12Q 1/68; G01N 33/567

(52) U.S. Cl. 435/6; 435/7.2

(57) ABSTRACT

Assays for screening for compounds that act as agonists, antagonists, or inverse agonists of CKβ8-1 at the FPRL-1 receptor.

### CROSS-REFERENCE TO RELATED APPLICATIONS

This application claims benefit under 35 U.S.C. §119(e) of U.S. Provisional Application 60/412,026, filed Sep. 19, 2002, incorporated herein by reference.

### FIELD OF THE INVENTION

The present invention relates to screening assays for compounds that modulate the interaction between CKβ8-1 and the FPRL-1 receptor.

### BACKGROUND

CKβ8, or myeloid progenitor inhibitor factor-1 (MPIF-1), is a CC chemokine (MacPhee et al., 1998, J. Immunol., 161:6273; Youn et al., 1998, Blood, 91:3118). CKβ8 cDNA encodes a signal sequence of 21 amino acids followed by 99 amino acid residues (CKβ8 protein) or 116 amino acid residues (CKβ8-1 protein). CKβ8-1 is a splice variant form of CKβ8. CKβ8 activates monoctyes, eosinophils, neutrophils, osteoclast precursors and lymphocytes. It also causes suppression of colony formation by progenitor cells found in human bone marrow. CKβ8 and CKβ8-1 are known to mediate some of their effects by activation via the CCR1 receptor.

G protein coupled receptors (GPCRs) constitute a family of proteins sharing a common structural organization characterized by an extracellular N-terminal end, seven hydrophobic alpha helices putatively constituting transmembrane domains and an intracellular C-terminal domain. GPCRs bind a wide variety of ligands that trigger intracellular signals through the activation of transducing G proteins (Caron et al., 1993, Rec. Prog. Horm. Res., 48:277-290; Freedman et al., 1996, Rec. Prog. Horm. Res., 51:319-353).

More than 300 GPCRs have been cloned thus far. Roughly 50-60% of all clinically relevant drugs are thought to act by modulating the functions of various GPCRs (Gudermann et al., 1995, J. Mol. Med., 73:51-63).

Among the GPCRs that have been identified and cloned is the formyl peptide receptor-like-1 (FPRL-1) receptor (Murphy et al., 1992, J. Biol Chem, 267:7637; Ye et al., 1992, Biochem. Biophys. Res. Comm., 184:582. This has been reported to be a low potency formyl-peptide receptor and a receptor for Lipoxin A4 and several other ligands (Klein et al., 1998, Nature Biotechnology, 16:1334-1337; Le et al., 2002, Int. Immunopharmacol., 2:1-13).

### SUMMARY

The present invention provides methods for identifying compounds that modulate the binding of CKβ8-1 to the FPRL-1 receptor comprising providing cells that express FPRL-1 receptor, or a functional fragment or variant thereof, contacting the cells with CKβ8-1, or a functional fragment or variant thereof, in the presence or absence of a test compound, and measuring a signal that is indicative of receptor activation, wherein an alteration to the signal in the presence of a compound identifies the tested compound as a compound that modulates the binding of CKβ8-1 to the FPRL-1 receptor.

The present invention also provides methods for identifying compounds that modulate the binding of CKβ8-1 to the FPRL-1 receptor comprising providing the FPRL-1 receptor or functional fragment or variant thereof, contacting the FPRL-1 receptor, or functional fragment or variant thereof, with CKβ8-1 or functional fragment or variant thereof in the presence or absence of a test compound, and measuring the amount of CKβ8-1 or functional fragment or variant thereof that forms a complex with the FPRL-1 receptor or functional fragment or variant thereof, wherein an alteration to the amount of the complex formed in the presence of the test compound identifies the compound as a compound that modulates binding of CKβ8-1 to FPRL-1 receptor.

The present invention also provides methods of distinguishing a FPRL-1 receptor agonist or antagonist comprising measuring a cell stimulating activity through a FPRL-1 receptor determined from contacting a compound with a cell expressing a FPRL-1 receptor or functional fragment or variant thereof (test screen), and comparing the results to a control screen wherein the cell does not express the FPRL-1 receptor or functional fragment or variant thereof, wherein said compound having cell stimulating activity in the test screen but not the control screen indicates that the test compound is a FPRL-1 receptor agonist, and contacting CKβ8-1 or functional fragment or variant thereof and a test compound with a cell expressing a FPRL-1 receptor or functional fragment or variant thereof (test screen), and comparing the results to a control screen wherein the cell does not express the FPRL-1 receptor or functional fragment or variant thereof, wherein a decrease in cell stimulating activity by CKβ8-1 or functional fragment or variant thereof in the test screen but not the control screen indicates that the test compound is a FPRL-1 receptor antagonist.

The invention also provides methods of screening for compounds that modulate binding of CKβ8-1 to the FPRL-1 receptor, comprising comparing the amount of CKβ8-1 or functional fragment or variant thereof bound to FPRL-1 receptor or functional fragment or variant thereof in steps (a) and (b), where step (a) comprises contacting CKβ8-1 or functional fragment or variant thereof with the FPRL-1 receptor or functional fragment or variant thereof, and step (b) comprises contacting CKβ8-1 or functional fragment or variant thereof and a test compound with the FPRL-1 receptor or functional fragment or variant thereof, wherein an alteration in the amount of CKβ8-1 or functional fragment or variant thereof bound to FPRL-1 receptor or functional fragment or variant thereof in step (b) indicates that the test compound modulates the binding of CKβ8-1 to the FPRL-1 receptor.

The invention also provides methods of screening for compounds that inhibit binding of CKβ8-1 to FPRL-1 receptor, comprising comparing the amount of CKβ8-1 or functional fragment or variant thereof bound to FPRL-1 receptor or functional fragment or variant thereof in steps (a) and (b), where step (a) comprises contacting CKβ8-1 or functional fragment or variant thereof with the FPRL-1 receptor or functional fragment or variant thereof, and step (b) comprises contacting CKβ8-1 or functional fragment or variant thereof and a test compound with the FPRL-1 receptor or functional fragment or variant thereof, wherein a decrease in CKβ8-1 or functional fragment or variant thereof binding in step (b) indicates that the test compound inhibits binding of CKβ8-1 to the FPRL-1 receptor.

The invention also provides methods of identifying a compound that modulates binding of CKβ8-1 to FPRL-1 receptor, comprising contacting FPRL-1 receptor or functional fragment or variant thereof with CKβ8-1 or functional fragment or variant thereof in the presence or absence of a test compound, and comparing the amount of binding between CKβ8-1 or functional fragment or variant thereof and the FPRL-1 receptor or functional fragment or variant thereof in the presence or absence of the test compound, wherein an alteration in the amount of binding between CKβ8-1 or functional fragment or variant thereof and the FPRL-1 receptor or functional fragment or variant thereof in the presence of the test compound indicates that the test compound modulates binding between CKβ8-1 and the FPRL-1 receptor.

The invention also provides methods of identifying compounds that can bind to the FPRL-1 receptor, comprising incubating a cell expressing the FPRL-1 receptor or functional fragment or variant thereof with CKβ8-1 or functional fragment or variant thereof in the presence or absence of a compound, and detecting displacement of CKβ8-1 or functional fragment or variant thereof binding to the FPRL-1 receptor or functional fragment or variant thereof in the presence of the compound, wherein the displacement is indicative of a compound that binds the FPRL-1 receptor.

The invention also provides methods of determining if a test compound is an agonist, antagonist or inverse agonist of CKβ8-1, comprising incubating a cell expressing FPRL-1 or functional fragment or variant thereof with the test compound, measuring a signal indicative of receptor activation and comparing the measurement with a second measurement of a signal indicative of receptor activation obtained from incubations performed in the absence of the test compound, wherein the test compound is determined to be an agonist of CKβ8-1 if the signal indicative of receptor activation is higher in the presence of the test compound than in its absence, and wherein the test compound is determined to be an antagonist of CKβ8-1 if the signal indicative of receptor activation is lower in the presence of the test compound than in its absence.

### DETAILED DESCRIPTION

We have discovered that CKβ8-1 activates the FPRL-1 receptor at nanomolar concentrations and that the FPRL-1 receptor is coupled to the G protein G_{αi/o}. We have also discovered that CKβ8-1 selectively binds to FPRL-1 and induces chemotaxis of peripheral blood cells.

The interaction between CKβ8-1 and the FPRL-1 receptor can be harnessed in assays to identify compounds that modulate binding of CKβ8-1 and the FPRL-1 receptor, to identify compounds that modulate CKβ8-1 activation of the FPRL-1 receptor, and to identify compounds that are agonists, antagonists or inverse agonists of the FPRL-1 receptor. Compounds identified in such assays can be used as therapeutic agents in the treatment of inflammatory disorders and in Alzheimer's disease. Compounds that modulate the binding interaction between CKβ8-1 and the FPRL-1 receptor can be identified. Compounds that modulate the activation of the FPRL-1 receptor can be identified.

As used herein, the terms “modulate” or “modulates” in reference to binding include any measurable alteration to the binding interaction between CKβ8-1 to FPRL-1 receptor, including, but not limited to, the amount or quantity of binding, binding affinity, and binding efficiency. For example, compounds identified using assays and methods of the present invention may increase or decrease the amount of binding of CKβ8-1 to the FPRL-1 receptor. Compounds identified using assays and methods of the present invention may enhance or inhibit the rate of binding of CKβ8-1 to FPRL-1 receptor.

As used herein, the term “inhibit” in reference to binding of CKβ8-1 to FPRL-1 receptor means any measurable decrease in binding.

As used herein, the term “decrease” in reference to cell stimulating activity or in reference to binding of CKβ8-1 to FPRL-1 receptor means any measurable diminution of such cell stimulating activity or binding activity.

As used herein, the term “increase” in reference to cell stimulating activity or in reference to binding of CKβ8-1 to FPRL-1 receptor means any measurable enhancement of such cell stimulating activity or binding activity.

As used herein, the terms “contact” or “contacting” refers to any method of combining components, such as combining compounds and/or CKβ8-1 in culture medium containing cells expressing the FPRL-1 receptor, or combining compounds and/or CKβ8-1 in solutions containing the FPRL-1 receptor, which may or may not be bound to a substrate.

As used herein, the phrase “functional fragment” in reference to CKβ8-1 protein, refers to portions or fragments of CKβ8-1 protein that are functionally active in the assays of the present invention, i.e., are capable of binding to and/or activating the FPRL-1 receptor. Functional fragment also includes fusion proteins that contain portions of CKβ8-1 protein.

As used herein, the phrase “functional fragment” in reference to FPRL-1 receptor protein, refers to portions or fragments of FPRL-1 receptor protein that are functionally active in the assays of the present invention, i.e., are capable of binding to and/or being activated by the CKβ8-1 protein. Functional fragment also includes fusion proteins that contain portions of FPRL-1 receptor.

As used herein, the term “variant” in reference to either CKβ8-1 protein or FPRL-1 receptor protein includes proteins having amino acid modifications, mutations, deletions, or insertions and other protein modifications that retain functionality in the assays of the present invention.

One skilled in the art can readily determine whether a protein or peptide is a functional fragment of CKβ8-1 or FPRL-1 receptor by examining its sequence and testing for binding and/or activation activity without undue experimentation. Truncated versions of CKβ8-1 or FPRL-1 receptor and fusion proteins containing portions of CKβ8-1 or FPRL-1 receptor may be prepared and tested using routine methods and readily available starting material.

As used herein, the term “heterologous” in reference to the FPRL-1 receptor gene means any non-endogenous FPRL-1 receptor gene, for example, one that has been introduced or transfected into a cell, which includes FPRL-1 receptor genes from different species or organisms than the cell and recombinant FPRL-1 receptor genes from the same species or organism as the cell.

Examples of signals that can be measured in assays of the present invention and which serve as indicators of receptor activation or indicators of cell stimulating activity include, but are not limited to, intracellular phospholipase C (PLC) activity, phospholipase A (PLA) activity, adenylyl cyclase activity, neutrophil chemotaxis, intracellular concentration of calcium in the cell, and opening and closing of ion channels. Many other methods of measuring receptor activation and cell stimulation are known to those skilled in the art and can be used in the assays of the present invention.

One aspect of the present invention is directed to assays for screening for compounds with the ability to modulate the binding of CKβ8-1 to the human FPRL-1 receptor. In some embodiments, cells expressing FPRL-1 receptor are used in conjunction with CKβ8-1 in screening assays designed to identify compounds that modulate CKβ8-1FPRL-1 binding. Cells expressing the FPRL-1 receptor can be incubated with CKβ8-1 and a test compound. The extent to which the binding of CKβ8-1 is displaced by the test compound is then determined. Radioligand assays or enzyme-linked immunosorbent assays may be performed in which either CKβ8-1 or the test compound is detectably labeled.

In some embodiments, cells expressing FPRL-1 receptor are used in assays to screen compounds that modulate CKβ8-1/FPRL-1 binding. Any cell type in which FPRL-1 receptor is expressed or can be engineered to be expressed can be used. Any cell type in which receptor binding and/or receptor activation can be measured may be used in the assays of the invention. By way of non-limiting examples, the assay may utilize mammalian cells (including, but not limited to, human, hamster, mouse, rat, or monkey) or non-mammalian cells such as amphibian (e.g., frog) or fish cells. Cell lines that may be used in the assays of the invention include, but are not limited to, HEK-293s (human embryonic kidney), CHO (Chinese hamster ovary), LTk- (murine fibroblasts lacking cytosolic deoxythymidine kinase (dTK)), HeLa, BALB/c-3T3, Xenopus oocytes; melanophores (cells from fish and amphibians) may also be used. In some embodiments, HEK-293s cells expressing the G protein G_{α16} are used.

In some embodiments, a recombinant cell expressing a heterologous FPRL-1 receptor from a heterologous gene expression construct is used. Any species of FPRL-1 receptor may be used, including, but not limited to a mammalian FPRL-1 receptor, including human, rodent, murine, rat, guinea pig, mouse, hamster, rhesus, cynomologous monkey, and porcine. The FPRL-1 receptor protein may be a fusion protein or may have variation in amino acid sequence, including deletions, insertions, mutations, and polymorphisms.

Another aspect of the invention relates to methods of determining if a test compound is an agonist, an antagonist, or an inverse agonist of CKβ8-1 binding based upon a functional assay. In some embodiments, assays are carried out by incubating a cell expressing FPRL-1 receptor with a test compound and determining whether intracellular phospholipase C activity, adenyl cyclase activity, or intracellular calcium concentrations are modulated. Results can be compared with controls wherein incubations are performed in a similar manner but in the absence of the test compound. Functional assays of this type can be performed in conjunction with binding assays, including those described herein. In some embodiments, the cell used in functional assays is a recombinant cell that has been transformed with a heterologous FPRL-1 gene.

Inverse agonists reduce phospholipase C activity or intracellular calcium levels, particularly if assays are performed in the presence of a fixed amount of CKβ8-1. Antagonists block binding of CKβ8-1 to the receptor but do not produce the opposite response in terms of phospholipase C activity or intracellular calcium that is the hallmark of an inverse agonist.

In some embodiments of the invention, FPRL-1 is recombinantly expressed in cells from a heterogenous or heterologous gene. The FPRL-1 receptor can be cloned as described by Murphy et al., 1992, supra. The FPRL-1 coding sequence can be incorporated into an expression vector with a promoter and other regulatory elements that will be active and appropriate for expression in the particular cell type used (see, Sambrook et al., eds., Molecular Cloning: A Laboratory Manual (2^{nd} ed.), Cold Spring Harbor. Laboratory Press, Cold Spring Harbor, N.Y. (1989)). In some embodiments, mammalian cells are used. Examples of promoters that may be used for expression in mammalian cells, include, but are not limited to, the mouse metallothionein I gene promoter (Hamer et al., 1982, J. Mol. Appl. Gen., 1:273-288), the immediate-early and TK promoter of herpes virus (Yao et al., 1995, J. Virol., 69:6249-6258, McKnight, 1982, Cell, 31:355-365); the SV40 virus early promoter (Benoist et al., 1981, Nature, 290:304-310), and the CMV promoter (Boshart et al., 1985, Cell, 41:521-530). Vectors may also include enhancers and other regulatory elements.

Expression vectors can be introduced into cells by methods well known to the art, including, but not limited to, calcium phosphate precipitation, microinjection, electroporation, liposomal transfer, viral transfer, or particle-mediated gene transfer.

In some embodiments the FPRL-1 receptor is used to screen for compounds that mimic the action of CKβ8-1 (agonists).

In some embodiments the FPRL-1 receptor is used to screen for compounds that antagonize the action of CKβ8-1 (antagonists).

In some embodiments the human FPRL-1 receptor is used.

In some embodiments human CKβ8-1 is used.

In some embodiments the form of CKβ8-1 that is used is the amino-terminally truncated form, CKβ8-1 (aa46-137).

Cells can be selected and assayed or examined for the expression of FPRL-1 according to standard procedures and techniques known to the art, including, but not limited to Northern blotting analysis.

In some embodiments, CKβ8-1 and cells expressing the FPRL-1 receptor are used in assays to determine whether test compounds have any effect on binding between CKβ8-1 and the FPRL-1 receptor. A wide variety of different types of assays can be performed using standard methods known to those of skill in the art. For example, in radioligand binding assays, cells expressing FPRL-1 are incubated with CKβ8-1 and with a compound being tested for binding activity. In some embodiments, the source of FPRL-1 is recombinantly transformed HEK-293s cells. In some embodiments, other cells types that do not express other proteins that strongly bind CKβ8-1 are utilized. Such cell types can easily be determined by performing binding assays on cells transformed with FPRL-1 and comparing the results obtained with those obtained using their non-transformed counterparts.

In some embodiments of the invention, functional assays, such as mobilization of intracellular calcium, are carried out using a FLIPR (Fluorescent Imaging Plate Reader) detection system.

In some embodiments of the invention, CKβ8-1 is iodinated and used as a tracer in radioligand binding assays on whole cells or membranes. Other assays that can be used include, but are not limited to, the GTPγS assay, adenylyl cyclase assays, assays measuring inositol phosphates, and reporter gene assays (e.g., those utilizing luciferase, aqueorin, alkaline phosphatase, etc.).

Assays may be performed using either intact cells or membranes prepared from the cells (see e.g., Wang et al., Proc. Natl. Acad. Sci. U.S.A. 90:10230-10234 (1993)). In some embodiments, membranes or whole cells are incubated with CKβ8-1 and with a preparation of the compound being tested. After binding is complete, the receptor is separated from the solution containing the ligand and test compound, e.g., by filtration, and the amount of binding that has occurred is determined. In some embodiments, the ligand used is detectably labeled with a radioisotope such as, for example, ¹²⁵I. Other types of labels can also be used, including, but not limited to, the following fluorescent labeling compounds: fluorescein, isothiocynate, rhodamine, phycoerythrin, phycocyanin, allophycocyanin o-phthaldehyde and fluorescamine. Chemiluminescent compounds can also be used with the assays of the invention, including, but not limited to, luminol, isoluminol, theromatic of acridinium ester, imidazole, acridinium salt, and oxalate ester.

In some embodiments of the invention, assays are performed in a cell-free environment, such as, for example, where only the binding interaction between CKβ8-1 and the FPRL-1 receptor is being examined. In such cell-free or in vitro binding assays FPRL-1 or CKβ8-1 may be bound to a support.

In some embodiments of the invention, assays are carried out wherein the compound is tested at different concentrations and the signal measured at these different concentrations permits the binding affinity of the compounds to be determined.

Nonspecific binding may be determined by carrying out the binding reaction in the presence of a large excess of unlabeled ligand. For example, labeled CKβ8-1 may be incubated with receptor and test compound in the presence of a thousand-fold excess of unlabeled CKβ8-1. Nonspecific binding should be subtracted from total binding, i.e., binding in the absence of unlabeled ligand, to arrive at the specific binding for each sample tested. Other steps such as washing, stirring, shaking, filtering and the like may be included in the assays as necessary. Typically, wash steps are included after the separation of membrane-bound ligand from ligand remaining in solution and prior to quantitation of the amount of ligand bound, e.g., by counting radioactive isotope. The specific binding obtained in the presence of test compound is compared with that obtained in the presence of labeled ligand alone to determine the extent to which the test compound has displaced receptor binding.

In performing binding assays, artifacts may falsely make it appear that a test compound is interacting with receptor when, in fact, binding is being inhibited by some other mechanism. Such artefact-generated false signals can be dealt with in a number of ways known to those of skill in the art. For example, the compound being tested can be placed in a buffer which does not itself substantially inhibit the binding of CKβ8-1 to the FPRL-1 receptor, and compounds can be tested at several different concentrations. Preparations of test compounds can be examined for proteolytic activity and antiproteases can be included in assays. Additionally, compounds that are identified as displacing the binding of CKβ8-1 to FPRL-1 receptor can be reexamined in a concentration range sufficient to perform a Scatchard analysis on the results. This type of analysis is well known in the art and can be used for determining the affinity of a test compound for a receptor (see e.g., Ausubel et al., eds., Current Protocols in Molecular Biology, 11.2.1-11.2.19, John Wiley & Sons, New York, N.Y. (1993); Work et al., eds., Laboratory Techniques in Biochemistry and Molecular Biology, NY (1978)). Computer programs can be used to assist in the analysis of results (e.g., Munson, 1983, Methods Enzymol., 92:543-577).

Depending upon their effect on the activity of the receptor, agents that inhibit the binding of CKβ8-1 to receptor may be either agonists or antagonists. Activation of receptor may be monitored using a number of different methods. For example, phospholipase C assays may be performed by growing cells in wells of a microtiter plate and then incubating the wells in the presence or absence of test compound total inositol phosphates (IP) may then be extracted in resin columns, and resuspended in assay buffer. Assay of IP thus recovered can be carried out using any method for determining IP concentration. Typically, phospholipase C assays are performed separately from binding assays, but it is also possible to perform binding and phospholipase C assays on a single preparation of cells.

Receptor activation can also be determined based upon a measurement of intracellular calcium concentration. Many types of assays for determining intracellular calcium concentrations are well known to the art and can be employed in the methods of the invention. For example, transformed HEK-293s can be grown to confluence on glass cover slides. After rinsing, the cells can be incubated in the presence of an agent such as Fluo-3, Fluo-4, or FURA-2 AM (Molecular Probes, Eugene, Oreg.). After rinsing and further incubation, calcium displacement can be measured using a photometer.

Assays that measure the intrinsic activity of the receptor, such as those based upon inositol phosphate measurement, can be used to determine the activity of inverse agonists. Unlike antagonists that block the activity of agonists but produce no activity on their own, inverse agonists produce a biological response diametrically opposed to the response produced by an agonist. For example, if an agonist promoted an increase in intracellular calcium, an inverse agonist would decrease intracellular calcium levels.

The radioligand and cell activation assays described herein provide examples of the types of assays that can be used for determining whether a particular test compound alters the binding of CKβ8-1 to the human FPRL-1 receptor and acts as an agonist or antagonist. There are many variations on these assays that are compatible with the present invention. Such assays can involve the use of labeled antibodies as a means for detecting CKβ8-1 that has bound to FPRL-1 receptor or may take the form of the fluorescent imaging plate reader assays.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### CKβ8-1 Induces Mobilization of Intracellular Ca²⁺ in FPRL-1 Expressing Cells

Expression of Human FPRL-1

HEK-293s cells expressing the G_{α16} protein (Molecular Devices, Sunnyvale, Calif.), or wild type cells, were transfected with a mammalian expression construct coding for the human FPRL-1 (pGENIRESneo vector) using FuGENE (Roche Diagnostics Corp, Indianapolis, Ind.). A stable receptor pool of FPRL-1 was developed by applying an antibiotic selection (G418, 1 mg/ml) and the cells were maintained in this selection medium. The expression and functional linkage of FPRL-1 was assessed by assaying the intracellular Ca²⁺ concentration ([Ca²⁺]ᵢ) using W-peptide (Trp-Lys-Tyr-Val-Met-NH2 (WKYMVM)) (SEQ ID NO:1) or its isoform (Trp-Lys-Tyr-Val-D-Met-NH2) (WKYMVm)) from Phoenix Pharmaceuticals, Inc. (Belmont, Calif.).

Ligands

In order to identify the ligand of human FPRL-1, a collection of peptide and non-peptide ligands was obtained from commercial sources (Sigma-Aldrich Corp. (St. Louis, Mo.), CalBiochem (San Diego, Calif.), American Peptide Company, Inc. (Sunnyvale, Calif.), Bachem Bioscience Inc. (King of Prussia, PA), Sigma-RBI (Natick, Mass.), R&D Systems (Minneapolis, Minn.), Phoenix Pharmaceuticals, Inc. (Belmont, Calif.)). The compounds were dissolved in water/DMSO at 3 μM and placed in 96-well microplates. A total of 2500 compounds (peptides and non-peptides) were prepared and tested.

Assay

A functional assay was performed with FLIPR (Fluorescent Imaging Plate Reader, Molecular Devices, Sunnyvale, Calif.) using the fluorescent calcium indicator Fluo-3 (Molecular Probes, Inc., Eugene, Oreg.) on a 96-well platform. HEK-293s cells expressing the human FPRL-1 receptor (and G_{α16} protein) or wild type cells only expressing the G_{α16} protein, were loaded with Fluo-3 as follows. Stable HEK-293s clones expressing FPRL-1 (+G_{α16}) and parental cells (+G_{α16}) were plated at a density of 20,000 cells/well in a 96-well plate. On the day of the experiment, the FPRL-1-transfected cells were loaded with fluorescent solution (Dulbecco's modified medium containing 4 μM Fluo-3 and 20% pluronic acid). The cells were incubated at 37° C. for one hour in a humidified chamber. Following the incubation step, cells were washed five times in Hanks' with 20 mM Hepes and 0.1% BSA (pH 7.4). The cells were analyzed using the FLIPR system to measure the mobilization of intracellular calcium in response to different compounds.

Results

HEK-293s and CHO cells endogenously express some GPCRs such as bradykinin and PACAP receptors, which were used as internal controls for assays. The background signal was established with all compounds in the parental HEK-293s or CHO cells transfected with the recombinant G_{α16} or G_{αqi5}. Cell lines transiently expressing human FPRL-1 were stimulated with compounds, and calcium responses were compared to parental cells. The functional assay with FLIPR gave calcium mobilization through the activation of FPRL-1 expressing cells (see results in Table 1A).

TABLE 1A

| | n = 1 | | n = 2 | |
|---|---|---|---|---|
| Ligand | EC₅₀ | Eₘₐₓ* | EC₅₀ | Eₘₐₓ* |
| ^{a}Rec human | 6.55 nM | 12063 | 0.52 nM | 21332 |
| CKβ8-1(aa46-137) | | | | |
| ^{b}Rec human | 0.43 nM | 8478 | 0.55 nM | 6332 |
| CKβ8-1(aa46-137) | | | | |
| ^{c}Rec human | 0.69 nM | 4124 | 0.14 nM | 6327 |
| CKβ8-1(aa46-137) | | | | |
| ^{d}Rec human | 2.9 nM | 4602 | 2.5 nM | 5949 |
| CKβ8-1(aa46-137) | | | | |
| ^{e}W-peptide (Control) | 0.57 nM | 13252 | 7.7 nM | 13326 |

^{a}HEK-293s cells stably expressing hFPRL-1 in G_{α16} background
^{b}HEK-293s transiently expressing hFPRL-1 in G_{α16} background
^{c}CHO cells transiently expressing hFPRL-1 in G_{α16} background
^{d}CHO cells transiently expressing hFPRL-1 in G_{αqi5} background
^{e}HEK-293s transiently expressing hFPRL-1 in G_{α16} background
*in relative fluorescent units (RFU)

Additional functional assays with FLIPR and G_{α16} gave calcium mobilization through the activation of FPRL-1 expressing cells in the presence of CKβ8-1 and other reported ligands (Le et al., 2002, Trends Immunol., 10:1-7) (see results in Table 1B).

TABLE 1B
Summary of pEC₅₀ and pIC₅₀ values of sCKβ8-1 and
known FPRL-1 ligands in calcium mobilization assay.

| | Intracellular Ca²⁺mobilization | |
|---|---|---|
| | pEC₅₀ (n = 3) | |
| Compound | CHO cells | HEK-293s cells |
| CKβ8-1 (aa46-137) | 9.13 ± 0.02 | 8.85 ± 0.07 |
| CKβ8 (aa46-120) | <5.0 | inactive |
| CKβ8-1 (aa22-137) | <5.7 | <5.7 |
| CKβ8 (aa22-120) | <5.0 | inactive |
| SHAAG peptide | 6.74 ± 0.23 | 7.15 ± 0.23 |
| Amyloid β protein (Aβ₄₂) | 6.09 ± 0.25 | <6.0 |
| Serum Amyloid A protein (SAA) | 6.88 ± 0.07 | <6.0 |
| Lipoxin A₄ (LXA₄) | <6.0 | <6.0 |
| Human Prion protein (hPrP) | <6.0 | <6.0 |
| W-peptide (WKYMVM) | 10.68 ± 0.25 | 9.56 ± 0.18 |
| W-peptide (WKYMVm) | n.t. | n.t. |

In Table 1B, The pEC₅₀ or pIC₅₀ values are given as mean±s.e. mean, and were calculated as −log of the EC₅₀ or −log of the IC₅₀ values (50% of the maximal compound effect); n.t.=not tested.

More than 1300 compounds were tested, including over 77 chemokines. An N-terminally truncated form of recombinant human CKβ8-1 (CKβ8-1 (aa46-137)) (R&D Systems, Minneapolis, Minn.), and W-peptide, a known FPRL-1 agonist, were the two most potent compounds to elicit a dose-dependent increase in the mobilization of intracellular calcium ([Ca²⁺]ᵢ) response in CHO or HEIC-293s cells co-expressing Gα₁₆ protein and FPRL-1 (see Table 1). Compounds reported in Table 1B did not elicit responses in non-transfected CHO or HEK-293s cells expressing either Gα₁₆ protein or other unrelated G-protein-coupled receptors. Moreover, in the absence of Gα₁₆, similar calcium mobilization responses were observed with CKβ8-1 (aa46-137) and W-peptide in CHO cells transiently expressing FPRL-1.

The results indicated that CKβ8-1 (aa46-137) was interacting with the transiently or stably expressed FPRL-1 receptor. Confirmation of this conclusion was obtained by the observation of a dose-response relationship with CKβ8-1 (aa46-137) in cells transfected with FPRL-1, but not in non-transfected cells or in cells transfected with other orphan receptors.

Interestingly, the short and long forms of CKβ8 (CKβ8 (aa46-120) and (CKβ8 (aa22-120)), as well as the long form of CKβ8-1 (CKβ8-1 (aa22-137)) (R&D Systems, Minneapolis, Minn.) displayed low potency (pEC₅₀<5.7) at FPRL-1 or were inactive (Table 1B).

These results suggested that the structural determinants of CKβ8-1 specificity for FPRL-1 might be the 17-amino acid peptide at the N-terminus, since the remaining sequence of the molecule is identical to CKβ8. To explore this hypothesis, we synthesized the 17-amino acid peptide (referred to as the “SHAAG peptide”), and determined its potency in cells co-expressing Gα₁₆ and FPRL-1. The SHAAG peptide: ₄₇LWRRKIGPQMTLSHAAG₆₃ (SEQ ID NO:2) (numbered to indicate the amino acid positioning within the CKβ8-1 protein sequence).

The SHAAG peptide was ˜60 to 200 times less potent at FPRL-1 as compared to CKβ8-1 (aa46-137), but ˜120 times more potent than the long form, CKβ8-1(aa22-137) (Table 1). In CHO and HEK-293s cells co-expressing Gα₁₆ and FPRL-1, other known FPRL-1 ligands (i.e., Aβ₄₂, SAA, and hPrP) were ˜200- to over 1000-fold less potent at FPRL-1 than CKβ8-1 (aa46-137), in agreement with published results (Le et al., 2002, Trends Immunol., 10:1-7), whereas the LXA₄ observed potency for FPRL-1 was low (pEC₅₀<6).

To eliminate the possibility that the low potency displayed by the full-length recombinant CKβ8-1 (aa22-137) at FPRL-1 was due to a misfolding during synthesis and/or degradation during the purification process, we measured [Ca²⁺]_{I}, release in cells stably expressing CCR1, and confirmed the biological activity of the samples used (Table 2).

TABLE 2
CKβ8-1 (aa22-137) is active on CCR1
stably expressed in HEK-293s cells.

| Compound | pEC₅₀ (n = 4) |
|---|---|
| CKβ8-1 (22-137) | 7.42 ± 0.03 |
| CKβ8-1 (46-137) | 8.14 ± 0.19 |
| CKβ8 (22-120) | 8.42 ± 0.07 |
| CKβ8 (46-120) | 8.88 ± 0.12 |
| RANTES | 9.57 ± 0.06 |

In Table 2, the pEC₅₀ values are given as mean±s.e. mean, and were calculated as−log of the EC₅₀ values.

Long forms CKβ8 (aa22-120) and CKβ8-1 (aa22-137) had been shown to potently activate CCR1 (Youn et al., 1998, Blood, 91:3118-3126). RANTES, a CCR1 agonist, produced a pEC₅₀ value of 9.57±0.06, in agreement with published results (Chou et al., 2002, Br. J. Pharmacol., 137:663-675). The rank order of potency of CKβ8, CKβ8-1 and of the N-terminally truncated forms at inducing calcium flux via CCR1 was as follows: CKβ8 (aa46-120)>CKβ8 (aa22-120)>CKβ8-1 (aa46-137)>CKβ8-1 (aa22-137) (Table 2).

These results indicated that, CKβ8-1 (aa22-137) is an active compound, and its potency is ˜200 to 300 fold lower at FPRL-1 than, at the CCR1 receptor.

### Example 2

### FPRL-1 is a G_{αi/o} Coupled Receptor

To determine which G_{α} protein is involved in the stimulation of PLCβ by FPRL-1, we tested if the Ca²⁺ mobilization pathway is stimulated by FPRL-1 ligands in parental CHO-K1 cell lines transiently expressing that receptor. Only W-peptide and CKβ8-1 (aa46-137), gave a consistent calcium response. The latter cells, transiently expressing FPRL-1, were treated with pertussis toxin (PTX). Pertussis toxin (PTX) pre-treatment completely abolished the dose-response dependent W-peptide and CKβ8-1-induced Ca²⁺ response, suggesting the involvement of G_{αi/o} and not G_{αq} protein in the mobilization of intracellular Ca²⁺. To assess the viability of cells, SLC1 expressing CHO-K1 cells were treated with PTX and incubated with melanin-concentrating hormone (MCH). In accordance with proposed dual coupling (Saito et al., 1999, Nature, 400:265-269) (G_{αi}/G_{αq}) PTX treatment partially blocked the MCH-induced Ca²⁺ response.

### Example 3

### CKβ8-1 (aa46-137) Inhibits the Adenylyl Cyclase Pathway

To further demonstrate the involvement of Gαᵢ/Gαₒ protein in FPRL-1 signalling pathway, the inhibition of forskolin-stimulated cAMP accumulation in CHO cells was assessed. CKβ8-1 alone, failed to inhibit basal cAMP levels but did inhibit, in a dose-dependent manner, the forskolin-stimulated cAMP accumulation (see Table 3). The pIC₅₀ values for W-peptide and its isoform for inhibition of forskolin-stimulated cAMP accumulation are in accordance with published data (Christophe et al., 2001, J. Biol. Chem., 276:2585-2593). Non-transfected CHO cells, or CHO cells expressing an unrelated GPCR were treated with the same range of agonist concentrations and exhibited no inhibition of cAMP accumulation. CKβ8-1 (aa46-137) and W-peptide yielded similar pIC₅₀ values in CHO cells stably expressing FPRL-1. These data confirm that FPRL-1 is a Gα_{i/o}-protein-coupled receptor, and is potently activated by CKβ8-1.

TABLE 3
pEC₅₀ and pIC₅₀ values of CKβ8-1 (aa46-137) and
known FPRL-1 ligands in various functional assays.

| | | [¹²⁵I]-W-peptide |
|---|---|---|
| | | (WKYMVm) |
| | Adenylyl cylase | Displacement |
| | pIC₅₀ | pIC₅₀ (n = 3) |
| Compound | CHO cells | CHO cells |
| CKβ8-1 (aa46-137) | 9.02 ± 0.20 (n = 4) | 7.97 ± 0.04 |
| CKβ8 (aa46-120) | inactive (n = 2) | inactive |
| CKβ8-1 (aa22-137) | n.t. | n.t. |
| CKβ8 (aa22-120) | n.t. | n.t. |
| SHAAG peptide | n.t. | n.t. |
| Amyloid β protein (Aβ₄₂) | 6.76; 5.90 (n = 2) | inactive |
| Serum Amyloid A protein | 6.38; 6.48 (n = 2) | <5.52 |
| (SAA) | | |
| Lipoxin A₄ (LXA₄) | <5.7 (n = 2) | inactive |
| Human Prion protein | inactive (n = 2) | inactive |
| (hPrP) | | |
| W-peptide (WKYMVM) | 10.38 ± 0.38 | 7.67 ± 0.06 |
| W-peptide (WKYMVm) | 11.87; 12.19 (n = 2) | 9.34 ± 0.08 |
| | | (Kd) |

The pIC₅₀ values are given as mean±s.e. mean, and were calculated as −log of the IC₅₀ values (50% of the maximal compound effect); n.t.=not tested.

### Example 4

### Efficient Displacement of [¹²⁵I]-W-peptide by CKβ81 (aa46137)

To characterise the binding properties of CKβ8-1, membranes prepared from CHO cells stably expressing FPRL-1 were incubated with the selective FPRL-1 ligand [¹²⁵I]-WKYMVm (Christophe et al., 2001, J. Biol. Chem, 276:2585-2593). The binding was specific and saturable for FPRL-1 using W-peptide. The observed K_{d} value for WKYMVm was 9.34±0.08, and CKβ8-1 was found to be the most effective, non-synthetic, agonist at competitively displacing [¹²⁵I]-WKYMVm (see Table 3), this was followed by SAA with a pIC₅₀ value of <5.52. In agreement with the low potency values observed in the calcium mobilization assay, other tested compounds did not displace the labelled ligand. Collectively, the data presented clearly demonstrate the ability of CKβ8-1 to bind to and activate the FPRL-1 receptor with high efficacy and potency, and supports the role of CKβ8-1 as a physiological and functional ligand for FPRL-1.

### Example 5

### CKβ8-1 Induces Calcium Flux and Chemotaxis in Polymorpho-Nuclear Leukocytes (PMNs)

Neutrophils play a pivotal role in the innate immune response to infection. Since these cells express FPRL-1, we evaluated the effect of CKβ8-1 (aa46-137) on PMNs calcium mobilization using the FLIPR system. CKβ8-1 dose-dependently increased the mobilization of [Ca²⁺]ᵢ. The rank order of potency for FPRL-1 ligands in PMNs was as follows: W-peptide>CKβ8-1 (aa46-137)≧MMK-1 (Table 4). Interleukin-8 (IL-8), known to activate CXCR1 and CXCR2 receptors, induced a dose-dependent calcium response indicating the integrity of the PMNs preparation.

TABLE 4
[Ca²⁺]ᵢ mobilization in PMNs was
measured in response to ligands shown.

| Compound | pEC50 |
|---|---|
| W-peptide | 9.58 ± 0.06 (n = 6) |
| CKβ8-1 | 7.42 ± 0.08 (n = 4) |
| SAA | <5.7 (n = 3) |
| MMK-1 | 7.17 ± 0.07 (n = 3) |
| IL-8 | 8.76 ± 0.24 (n = 3) |
| LXA4 | inactive (n = 4) |
| MIP-1α | inactive (n = 4) |
| F-peptide | inactive (n = 4) |

The physiological relevance of CKβ8-1 (aa46-137) as a ligand for FPRL-1 was assessed by PMNs chemotaxis experiments. CKβ8-1 (aa46-137), MMK-1 and W-peptide (WKYMVm) induced the migration of PMNs at concentrations ranging from 1 pM to 20 μM. The maximum percentage of cell migration produced by sCKβ8-1 was reached at 1 μM, 12 μM with MMK-1 and 100 nM with W-peptide.

The cell migration data demonstrates the ability of CKβ8-1 to activate human PMNs and suggests that this activity is mediated via FPRL-1 receptor endogenously expressed in these cells. To demonstrate the specificity of CKβ8-1 for FPRL-1, human PMNs were pretreated in the presence or absence of a monoclonal anti-FPRL-1 antibody, and calcium mobilization in response to CKβ8-1 was measured. In PMNs, antibody pretreatment reduced the [Ca^{2+]}ᵢ mobilization by 80-90% when incubated with CKD8-1 (aa46-137). Similar responses were obtained in HEK-293s cells stably co-expressing Gα₁₆ and FRL-1.

Collectively, the data confirm that the effect produced by CKβ8-1 in human PMNs is mediated by FPRL-1.

The foregoing examples are meant to illustrate the invention and are not to be construed to limit the invention in any way. Those skilled in the art will recognize modifications that are within the spirit and scope of the invention.

We claim:

1. A method for identifying compounds that modulate binding of CKβ8-1 to FPRL-1 receptor, said method comprising:
providing cells expressing FPRL-1 receptor or functional fragment or variant thereof;
contacting said cells with CKβ8-1 or a functional fragment or variant thereof, in the presence or absence of a compound; and
measuring a signal indicative of receptor activation;
where an alteration to said signal in the presence of a compound identifies said compound as a compound that modulates binding of CKβ8-1 to FPRL-1 receptor.

2. The method of claim 1, wherein the FPRL-1 receptor is expressed from a heterologous FPRL-1 receptor gene.

3. The method of claim 1, wherein the FPRL-1 receptor is mammalian.

4. The method of claim 3, wherein the FPRL-1 receptor is human.

5. The method of claim 1, wherein said cells are mammalian cells.

6. The method of claim 5, wherein the cells are human cells.

7. The method of claim 1, wherein said measuring is performed using a FLIPR assay.

8. The method of claim 1, wherein the signal measured is modulation of intracellular phospholipase C activity, intracellular adenyl cyclase activity or intracellular calcium concentration.

9. The method of claim 1, wherein the CKβ8-1 is CKβ8-1 (aa46-137).

10. A method for identifying compounds that modulate the binding of CKβ8-1 to the FPRL-1 receptor, said method comprising:
providing the FPRL-1 receptor or functional fragment or variant thereof;
contacting the FPRL-1 receptor or functional fragment or variant thereof, with CKβ8-1 or functional fragment or variant thereof in the presence or absence of a compound; and
measuring the amount of CKβ8-1 or functional fragment or variant thereof that forms a complex with the FPRL-1 receptor or functional fragment or variant thereof;
where an alteration to the amount of said complex formed in the presence of said compound identifies said compound as a compound that modulates binding of CKβ8-1 to the FPRL-1 receptor.

11. The method of claim 10, wherein FPRL-1 receptor/CKβ8-1 complexes are isolated prior to measuring the amount of CKβ8-1 in said complexes.

12. The method of claim 11, wherein the CKβ8-1 is detectably labeled.

13. The method of claim 12, wherein the CKβ8-1 is radiolabled, fluorescently labeled, or chemiluminescently labeled.

14. The method of claim 13, wherein the CKβ8-1 is radiolabled.

15. The method of claim 10, wherein CKβ8-1 is bound to an enzyme, and measuring is carried out by enzyme-linked immunosorbent assay (ELISA).

16. The method of claim 10, wherein the FPRL-1 receptor, or functional fragment or variant thereof, is human.

17. The method of claim 10, wherein the CKβ8-1 is CKβ8-1 (aa46-137).

18. The method of claim 10, wherein the FPRL-1 receptor, or functional fragment or variant thereof, is provided as cells expressing the FPRL-1 receptor or functional fragment or variant thereof, or is provided as membranes prepared from said cells.

19. The method of claim 18, wherein the FPRL-1 receptor, or functional fragment or variant thereof, is expressed from a heterologous FPRL-1 receptor gene.

20. The method of claim 18, wherein said cells are mammalian cells.

21. The method of claim 20, wherein the cells are human cells.

22. A method of screening for a FPRL-1 receptor agonist or antagonist comprising measuring a cell stimulating activity through a FPRL-1 receptor determined from the following steps a) and b);
a) contacting a compound with a cell expressing a FPRL-1 receptor or functional fragment or variant thereof (test screen), and comparing the results to a control screen wherein the cell does not express the FPRL-1 receptor or functional fragment or variant thereof, wherein said compound having cell stimulating activity in the test screen but not the control screen indicates that the test compound is a FPRL-1 receptor agonist,
b) contacting CKβ8-1 or functional fragment or variant thereof and a test compound with a cell expressing a FPRL-1 receptor or functional fragment or variant thereof (test screen), and comparing the results to a control screen wherein the cell does not express the FPRL-1 receptor or functional fragment or variant thereof, where a decrease in cell stimulating activity by CKβ8-1 or functional fragment or variant thereof in the test screen but not the control screen indicates that the test compound is a FPRL-1 receptor antagonist.

23. The method of claim 22, wherein the CKβ8-1 is CKβ8-1 (aa46-137).

24. The method of claim 23, wherein the cell stimulating activity is intracellular phospholipase C activity, intracellular adenylyl cyclase activity, or intracellular calcium concentration.

25. A method of screening for compounds that modulate binding of CKβ8-1 to FPRL-1 receptor, comprising comparing the amount of CKβ8-1 or functional fragment or variant thereof bound to FPRL-1 receptor or functional fragment or variant thereof in steps a) and b):
a) contacting CKβ8-1 or functional fragment or variant thereof with the FPRL-1 receptor or functional fragment or variant thereof;
b) contacting CKβ8-1 or functional fragment or variant thereof and a test compound with the FPRL-1 receptor or functional fragment or variant thereof;
where an alteration in the amount of CKβ8-1 or functional fragment or variant thereof bound to FPRL-1 receptor or functional fragment or variant thereof in step b) indicates that the test compound modulates binding of CKβ8-1 to the FPRL-1 receptor.

26. The method of claim 25, wherein the CKβ8-1 is CKβ8-1 (aa46-137).

27. A method of screening for compounds that inhibit binding of CKβ8-1 to FPRL-1 receptor, comprising comparing the amount of CKβ8-1 or functional fragment or variant thereof bound to FPRL-1 receptor or functional fragment or variant thereof in steps a) and b):
a) contacting CKβ8-1 or functional fragment or variant thereof with the FHRL-1 receptor or functional fragment or variant thereof;
b) contacting CKβ8-1 or functional fragment or variant thereof and a test compound with the FPRL-1 receptor or functional fragment or variant thereof;
where a decrease in CKβ8-1 or functional fragment or variant thereof binding in step b) indicates that the test compound inhibits binding of CKβ8-1 to the FPRL-1 receptor.

28. The method of claim 27, wherein the CKβ8-1 is CKβ8-1 (aa46-137).

29. A method of identifying a compound that modulates binding of CKβ8-1 to FPRL-1 receptor, comprising contacting FPRL-1 receptor or functional fragment or variant thereof with CKβ8-1 or functional fragment or variant thereof in the presence or absence of a test compound, and comparing the amount of binding between CKβ8-1 or functional fragment or variant thereof and the FPRL-1 receptor or functional fragment or variant thereof in the presence or absence of the test compound, where an alteration in the amount of binding between CKβ8-1 or functional fragment or variant thereof and the FPRL-1 receptor or functional fragment or variant thereof in the presence of the test compound indicates that the test compound modulates binding between CKβ8-1 and the FPR-1 receptor.

30. The method of claim 29, wherein the CKβ8-1 is CKβ8-1 (aa46-137).

31. A method of identifying a compound that binds FPRL-1 receptor, comprising incubating a cell expressing FPRL-1 receptor or functional fragment or variant thereof with CKβ8-1 or functional fragment or variant thereof in the presence or absence of a compound, and detecting displacement of CKβ8-1 or functional fragment or variant thereof binding to the FPRL-1 receptor or functional fragment or variant thereof in the presence of the compound, where displacement of said binding is indicative of a compound that binds the FPRL-1 receptor.

32. The method of claim 31, wherein the CKβ8-1 is CKβ8-1 (aa46-137).

33. A method of determining if a test compound is an agonist, antagonist or inverse agonist of CKβ8-1 comprising
a) incubating a cell expressing FPRL-1 or functional fragment or variant thereof with the test compound;
b) measuring a signal indicative of receptor activation; and
c) comparing the measurement in b) with a second measurement of a signal indicative of receptor activation obtained from incubations performed in the absence of the test compound, where the test compound is an agonist of CKβ8-1 if the signal indicative of receptor activation is higher in the presence of the test compound than in its absence, and wherein the test compound is an antagonist of CKβ8-1 if the signal indicative of receptor activation is lower in the presence of the test compound than in its absence.
